(19) United States
(12) Patent Application Publication
(10) Pub. No.: US 2016/0206212 A1
(43) Pub. Date: Jul. 21, 2016

(54) USER-WEARABLE DEVICES INCLUDING OPTICAL SENSORS WITH POWER SAVING FEATURES AND METHODS FOR USE THEREWITH

(71) Applicant: Salutron, Inc., Fremont, CA (US)

(72) Inventors: Yong Jin Lee, Palo Alto, CA (US); Yanqiu Wang, Fremont, CA (US)

(73) Assignee: SALUTRON, INC., Fremont, CA (US)

(21) Appl. No.: 14/598,765

(22) Filed: Jan. 16, 2015

(51) Int. Cl. A61B 5/0205; A61B 5/00

(52) U.S. Cl. CPC A61B 5/0205; A61B 5/6801; A61B 5/02416

(57) ABSTRACT

A battery powered user-wearable device includes a light source and a light detector and is configured to obtain at least two different types of physiological measurements using the light source and the light detector. During a first period of time, the user-wearable device is operated in accordance with a first operational mode that is used to obtain a first type of physiological measurement. In the first operational mode the light source is driven to emit pulses of light at a low frequency. During a second period of time, the user-wearable device is operated in accordance with a second operational mode that is used to obtain a second type of physiological measurement. In the second operational mode the light source is either driven to continually emit light or is driven to emit pulses of light at a high frequency. The first operational mode consumes less power than the second operational mode.

### BACKGROUND

User-wearable devices, such as activity monitors or actigraphs, have become popular as a tool for promoting exercise and a healthy lifestyle. Such user-wearable devices can be used, for example, to measure heart rate and/or other physiological measurements. Such user-wearable devices may also measure steps taken while walking or running and/or estimate an amount of calories burned. Additionally, or alternatively, a user-wearable device can be used to monitor sleep related metrics. User-wearable devices, such as smart watches, can additionally or alternatively be used to provide alerts to a user. Such user-wearable devices are typically battery operated. Because such user-wearable devices are often used to perform numerous functions that consume power, if not appropriately designed and operated the battery life of such devices can be relatively short, which is undesirable.

### BRIEF DESCRIPTION OF THE DRAWINGS

FIG. 1A depicts a front view of a user-wearable device, according to an embodiment.

FIG. 1B depicts a rear view of the user-wearable device of FIG. 1A, according to an embodiment.

FIG. 2 depicts a high level block diagram of electrical components of the user-wearable device introduced in FIGS. 1A and 1B, according to an embodiment.

FIG. 3 is a high level flow diagram that is used to summarize methods according to certain embodiments of the present technology.

FIG. 4 is another high level flow diagram that is used to summarize methods according to certain embodiments of the present technology.

FIG. 5 is a block diagram that is used to provide additional details of the optical sensor introduced in FIG. 1B, according to an embodiment.

FIG. 6 is a block diagram that is used to illustrate that different analog signal processing circuitry can be used to amplify and/or filter a light detection signal depending upon an operational mode.

### DETAILED DESCRIPTION

In the following detailed description, reference is made to the accompanying drawings that form a part hereof, and in which is shown by way of illustration specific illustrative embodiments. It is to be understood that other embodiments may be utilized and that mechanical and electrical changes may be made. The following detailed description is, therefore, not to be taken in a limiting sense. In the description that follows, like numerals or reference designators will be used to refer to like parts or elements throughout. In addition, the first digit of a reference number identifies the drawing in which the reference number first appears.

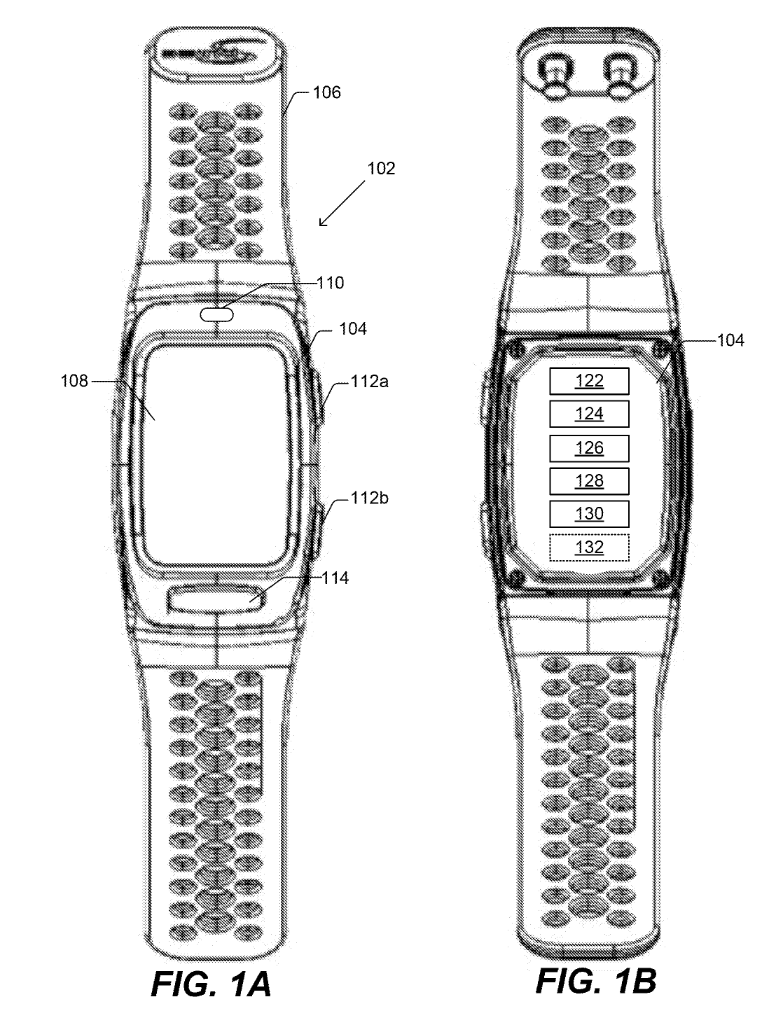

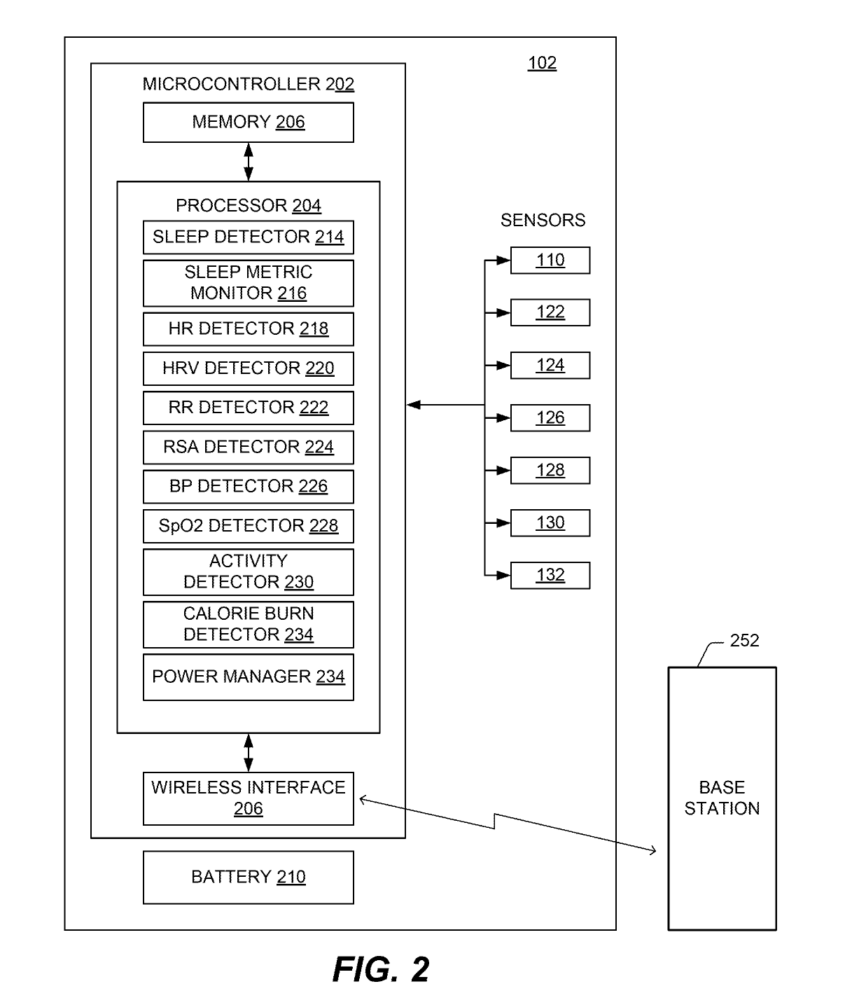

FIG. 1A depicts a front view of a user-wearable device 102, according to an embodiment. The user-wearable device 102 can be a standalone device which gathers and processes data and displays results to a user. Alternatively, the user-wearable device 102 can wirelessly communicate with a base station (252 in FIG. 2), which can be a mobile phone, a tablet computer, a personal data assistant (PDA), a laptop computer, a desktop computer, or some other computing device that is capable of performing wireless communication. The base station can, e.g., include a health and fitness software application and/or other applications, which can be referred to as apps. The user-wearable device 102 can upload data obtained by the device 102 to the base station, so that such data can be used by a health and fitness software application and/or other apps stored on and executed by the base station.

The user-wearable device 102 is shown as including a housing 104, which can also be referred to as a case 104. A band 106 is shown as being attached to the housing 104, wherein the band 106 can be used to strap the housing 104 to a user's wrist or arm. The housing 104 is shown as including a digital display 108, which can also be referred to simply as a display. The digital display 108 can be used to show the time, date, day of the week and/or the like. The digital display 108 can also be used to display activity and/or physiological metrics, such as, but not limited to, heart rate (HR), heart rate variability (HRV), respiratory sinus arrhythmia (RSA), calories burned, steps taken and distance walked and/or run. The digital display 108 can further be used to display sleep metrics, examples of which are discussed below. These are just examples of the types of information that may be displayed on the digital display 108, which are not intended to be all encompassing.

The housing 104 is also shown as including an outward facing ambient light sensor (ALS) 110, which can be used to detect ambient light, and thus, can be useful for detecting whether it is daytime or nighttime, as well as for other purposes. The housing 104 is further shown as including buttons 112a, 112b, which can individually be referred to as a button 112, and can collectively be referred to as the buttons 112. One of the buttons 112 can be a mode select button, while another one of the buttons 112 can be used to start and stop certain features. While the user-wearable device 102 is shown as including two buttons 112, more or less than two buttons can be included. The buttons 112 can additionally or alternatively be used for other functions. The housing 104 is further shown as including a forward facing ECG electrode 114, which is discussed below. This ECG electrode 114 can also function as an additional button.

In certain embodiments, the user-wearable device 102 can receive alerts from a base station (e.g., 252 in FIG. 2). For example, where the base station 252 is a mobile phone, the user wearable device 100 can receive alerts from the base station, which can be displayed to the user on the display 108. For a more specific example, if a mobile phone type of base station 252 is receiving an incoming phone call, then an incoming phone call alert can be displayed on the digital display 108 of the mobile device, which may or may not include the phone number and/or identity of the caller. Other types of alerts include, e.g., text message alerts, social media alerts, calendar alerts, medication reminders and exercise reminders, but are not limited thereto. The user-wearable device 102 can inform the user of a new alert by vibrating and/or emitting an audible sound.

FIG. 1B illustrates a rear-view of the housing 104 of the user-wearable device 102. Referring to FIG. 1B, the backside of the housing 104 includes an optical sensor 122, a capacitive sensor 124, a galvanic skin resistance sensor 126, an electrocardiogram (ECG) sensor 128 and a skin temperature sensor 130. It is also possible that the user-wearable device 102 includes less sensors than shown, more sensors than shown and/or alternative types of sensors. For example, the user-wearable device 102 can also include one or more type of motion sensor 132, which is shown in dotted line because it is likely completely encased with the housing 104.

In accordance with an embodiment, the optical sensor 122 includes both a light source and a light detector. The light source of the optical sensor 122 can include one or more light emitting diode (LED), incandescent lamp or laser diode, but is not limited thereto. While infrared (IR) light sources are often employed in optical sensors, because the human eye cannot detect IR light, the light source can alternatively produce light of other wavelengths. The light detector of the optical sensor 122 can include one or more one or more photoresistor, photodiode, phototransistor, photodarlington or avalanche photodiode, but is not limited thereto. The light source of the optical sensor 122 can be selectively driven to emit light. If an object (e.g., a user's wrist or arm) is within the sense region of the optical sensor 122, a large portion of the light emitted by the light source will be reflected off the object and will be incident on the light detector. The light detector generates a signal (e.g., a current) that is indicative of the intensity and/or phase of the light incident on the light detector, and thus, can be used to detect the presence of the user's wrist or arm. Where the signal generated by the light detector is a current signal, it can be converted to a voltage signal, if desired, using a transimpedance amplifier. The signal can be converted to a digital signal using an analog to digital converter. Additional analog and/or digital signal processing can be performed on such a signal. Regardless of whether the signal generated using the light detector is a current or voltage signal, or an analog or digital signal, such a signal can be referred to generally as a light detection signal. The optical sensor 122 may also use its light detector to operate as an ambient light detector. It is also possible that the optical sensor 122 not include a light source, in which case the optical sensor 122 can operate as an ambient light sensor, but not a proximity sensor. When operating as an ambient light sensor, the optical sensor 122 produces a signal having a magnitude that is dependent on the amount of ambient light that is incident on the optical sensor 122. It is expected that when a user is wearing the user-wearable device 102 on their wrist or arm, the light detector of the optical sensor 122 will be blocked (by the user's wrist or arm) from detecting ambient light, and thus, the signal produced the light detector will have a very low magnitude.

Additionally, or alternatively, the optical sensor 122 can also be used to detect heart rate (HR), heart rate variability (HRV), respiratory rate (RR) and/or respiratory sinus arrhythmia (RSA). More specifically, the optical sensor 122 can operate as a photoplethysmography (PPG) sensor, in which case, the optical sensor 122 can also be referred to as a PPG sensor. When operating as a PPG sensor, the light source of the optical sensor 122 emits light that is reflected or backscattered by user tissue, and reflected/backscattered light is received by the light detector of the optical sensor 122. In this manner, changes in reflected light intensity are detected by the light detector, which outputs a PPG signal indicative of the changes in detected light, which are indicative of changes in blood volume. The PPG signal output by the light detector can be filtered and amplified, and can be converted to a digital signal using an analog-to-digital converter (ADC), if the PPG signal is to be analyzed in the digital domain. Each cardiac cycle in the PPG signal generally appears as a peak, thereby enabling the PPG signal to be used to detect peak-to-peak intervals, which can be used to calculate heart rate (HR) and heart rate variability (HRV). Slow oscillations in a baseline of the PPG signal are due to changes in intrathoracic pressure due to respiration. Accordingly, respiration rate (RR) can also be determined based on the PPG signal. Further, if desired, a signal indicative of respiration can be produced based on the PPG signal, by filtering and/or performing other signal processing on the PPG signal. Further, this enables the PPG signal to be used to calculate a level or magnitude of respiratory sinus arrhythmia (RSA).

In accordance with certain embodiments, the optical sensor 122 includes a light source that emits light of two different wavelengths that enables the optical sensor 122 to be used as a pulse oximeter, in which case the optical sensor 122 can be used to non-invasively monitor the blood oxygen saturation (SpO2) of a user wearing the user-wearable device 102. For example, the optical sensor 122 can include one or more LED that emits red light (e.g., about 660 nm wavelength) and one or more further LED that emits infrared or near infrared light (e.g., about 940 nm wavelength), but is not limited thereto.

In accordance with an embodiment, the capacitive sensor 124 includes an electrode that functions as one plate of a capacitor, while an object (e.g., a user's wrist or arm) that is in close proximity to the capacitive sensor 124 functions as the other plate of the capacitor. The capacitive sensor 124 can indirectly measure capacitance, and thus proximity, e.g., by adjusting the frequency of an oscillator in dependence on the proximity of an object relative to the capacitive sensor 124, or by varying the level of coupling or attenuation of an AC signal in dependence on the proximity of an object relative to the capacitive sensor 124.

The galvanic skin resistance (GSR) sensor 126 senses a galvanic skin resistance. The galvanic skin resistance measurement will be relatively low when a user is wearing the user-wearable device 102 on their wrist or arm and the GSR sensor 126 is in contact with the user's skin. By contrast, the galvanic skin resistance measurement will be very high when a user is not wearing the user-wearable device 102 and the GSR sensor 126 is not in contact with the user's skin.

The ECG sensor 128 can be used to obtain an ECG signal from a user that is wearing the user-wearable device 102 on their wrist or arm (in which case the ECG sensor 128, which is an electrode, is in contact with the user's wrist or arm), and the user touches the front facing ECG electrode 114 with their other arm (e.g., with a finger of their other arm). Additionally, or alternatively, an ECG sensor can be incorporated into a chest strap that provides ECG signals to the user-wearable device 102. The skin temperature sensor 130 can be implemented, e.g., using a thermistor, and can be used to sense the temperature of a user's skin, which can be used to determine user activity and/or calories burned.

Depending upon implementation, heart rate (HR) and/or heart rate variability (HRV) can be determined based on signals obtained by the optical sensor 122 and/or the ECG sensor 128 (which can include the electrode 114). Additionally, respiration rate (RR) and/or respiratory sinus arrhythmia (RSA) level can be determined based on signals obtained by the optical sensor 122 and/or the ECG sensor 128 (which can include the electrode 114). One or more of HR, HRV, RR and/or RSA can be automatically determined periodically, in response to a triggering condition or event, at other specified times or based on a manual user action. For example, in a free living application, HR can be determined automatically during periods of interest, such as when a significant amount of activity is detected.

Additional physiologic metrics can also be obtained using the sensors described herein. For example, blood pressure can be determined from the PPG and ECG signals by determining a metric of pulse wave velocity (PWV) and converting the metric of PWV to a metric of blood pressure. More specifically, a metric of PWV can be determining by determining a time from a specific feature (e.g., an R-wave) of an obtained ECG signal to a specific feature (e.g., a maximum upward slope, a maximum peak or a dicrotic notch) of a simultaneously obtained PPG signal. An equation can then be used to convert the metric of PWV to a metric of blood pressure. To measure the metric of PWV, high temporal resolution is preferred.

In accordance with an embodiment the motion sensor 132 is an accelerometer. The accelerometer can be a three-axis accelerometer, which is also known as a three-dimensional (3D) accelerometer, but is not limited thereto. The accelerometer may provide an analog output signal representing acceleration in one or more directions. For example, the accelerometer can provide a measure of acceleration with respect to x, y and z axes. The motion sensor 132 can alternatively be a gyrometer, which provides a measure of angular velocity with respect to x, y and z axes. It is also possible that the motion sensor 132 is an inclinometer, which provides a measure of pitch, roll and yaw that correspond to rotation angles around x, y and z axes. For another example, the motion sensor 132 can include an e-Compass. It is also possible the user wear-able device 102 includes multiple different types of motion sensors, some examples of which were just described. Depending upon the type(s) of motion sensor(s) used, such a sensor can be used to detect the posture of a portion of a user's body (e.g., a wrist or arm) on which the user-wearable device 102 is being worn.

FIG. 2 depicts an example block diagram of electrical components of the user-wearable device 102, according to an embodiment. Referring to FIG. 2, the user-wearable device 102 is shown as including a microcontroller 202 that includes a processor 204, memory 206 and a wireless interface 208. It is also possible that the memory 206 and wireless interface 208, or portions thereof, are external the microcontroller 202. The microcontroller 202 is shown as receiving signals from each of the aforementioned sensors 110, 122, 124, 126, 128 and 130. The user-wearable device 102 is also shown as including a battery 210 that is used to power the various components of the device 102. While not specifically shown, the user-wearable device 102 can also include one or more voltage regulators that are used to step-up and or step-down the voltage provided by the battery 210 to appropriate levels to power the various components of the device 102.

Each of the aforementioned sensors 110, 122, 124, 126, 128, 130, 132 can include or have associated analog signal processing circuitry to amplify and/or filter raw signals produced by the sensors. It is also noted that analog signals produced using the aforementioned sensors 110, 122, 124, 126, 128, 130 and 122 can be converted to digital signals using one or more digital to analog converters (ADCs), as is known in the art. The analog or digital signals produced using these sensors can be subject time domain processing, or can be converted to the frequency domain (e.g., using a Fast Fourier Transform or Discrete Fourier Transform) and subject to frequency domain processing. Such time domain processing, frequency domain conversion and/or frequency domain processing can be performed by the processor 204, or by some other circuitry.

The user-wearable device 102 is shown as including various modules, including a sleep detector module 214, a sleep metric module 216, a heart rate (HR) detector module 218, a heart rate variability (HRV) detector module 220, a respiratory rate (RR) detector module 222, a respiratory sinus arrhythmia (RSA) detector module 224, a blood pressure (BP) detector module 226, and SpO2 detector module 228, an activity detector module 230, a calorie burn detector module 232 and a power manager module 234. The various modules may communicate with one another, as will be explained below. Each of these modules 214, 216, 218, 220, 222, 224, 226, 228, 230, 232 and 234 can be implemented using software, firmware and/or hardware. It is also possible that some of these modules are implemented using software and/or firmware, with other modules implemented using hardware. Other variations are also possible. In accordance with a specific embodiments, each of these modules 214, 216, 218, 220, 222, 224, 226, 228, 230, 232 and 234 is implemented using software code that is stored in the memory 206 and is executed by the processor 204. The memory 206 is an example of a tangible computer-readable storage apparatus or memory having computer-readable software embodied thereon for programming a processor (e.g., 204) to perform a method. For example, non-volatile memory can be used. Volatile memory such as a working memory of the processor 204 can also be used. The computer-readable storage apparatus may be non-transitory and exclude a propagating signal.

The sleep detector module 214, which can also be referred to simply as the sleep detector 214, uses signals and/or data obtained from one or more of the above described sensors to determine whether a user, who is wearing the user-wearable device 102, is sleeping. For example, signals and/or data obtained using the outward facing ambient light sensor (ALS) 110 and/or the motion sensor 132 can be used to determine when a user is sleeping. This is because people typically sleep in a relatively dark environment with low levels of ambient light, and typically move around less when sleeping compared to when awake. Additionally, if the user's arm posture can be detected from the motion sensor 132, then information about arm posture can also be used to detect whether or not a user is sleeping.

The sleep metric detector module 216, which can also be referred to as the sleep metric detector 216, uses information obtained from one or more of the above described sensors and/or other modules to quantify metrics of sleep, such as total sleep time, sleep efficiency, number of awakenings, and estimates of the length or percentage of time within different sleep states, including, for example, rapid eye movement (REM) and non-REM states. The sleep metric module 216 can, for example, use information obtained from the motion sensor 132 and/or from the HR detector 218 to distinguish between the onset of sleep, non-REM sleep, REM sleep and the user waking from sleep. One or more quality metric of the user's sleep can then be determined based on an amount of time a user spent in the different phases of sleep. Such quality metrics can be displayed on the digital display 108 and/or uploaded to a base station (e.g., 252) for further analysis.

The HR detector module 218, which can also be referred to simply as the HR detector 218, uses signals and/or data obtained from the optical sensor 122 and/or the ECG sensor 128 (which can include the electrode 114) to detect HR. For example, the optical sensor 122 can be used to obtain a PPG signal from which peak-to-peak intervals can be detected. For another example, the ECG sensor 128 (which can include the electrode 114) can be used to obtain an ECG signal, from which peak-to-peak intervals (e.g., Rwave-to-Rwave intervals) can be detected. The peak-to-peak intervals of a PPG signal or an ECG signal can also be referred to as beat-to-beat intervals, which are intervals between heart beats. Beat-to-beat intervals can be converted to HR using the equation HR=(1/beat-to-beat interval)*60. Thus, if the beat-to-beat interval=1 sec, then HR=60 beats per minute (bpm); or if the beat-to-beat interval=0.6 sec, then HR=100 bpm. The user's HR can be displayed on the digital display 108 and/or uploaded to a base station (e.g., 252) for further analysis.

The HRV detector module 220, which can also be referred to simply as the HRV detector 220, uses signals and/or data obtained from the optical sensor 122 and/or the ECG sensor 128 (which can include the electrode 114) to detect HRV. For example, in the same or a similar manner as was explained above, beat-to-beat intervals can be determined from a PPG signal obtained using the optical sensor 122 and/or from an ECG signal obtained using the ECG sensor 128 (which can include the electrode 114). HRV can be determined by calculating a measure of variance, such as, but not limited to, the standard deviation (SD), the root mean square of successive differences (RMSSD), or the standard deviation of successive differences (SDSD) of a plurality of consecutive beat-to-beat intervals. Alternatively, or additionally, obtained PPG and/or ECG signals can be converted from the time domain to the frequency domain, and HRV can be determined using well known frequency domain techniques. The user's HRV can be displayed on the digital display 108 and/or uploaded to a base station (e.g., 252) for further analysis.

The RR detector module 222, which can also be referred to simply as the RR detector 222, uses signals and/or data obtained from the optical sensor 122 to detect respiratory rate (RR). The RSA detector module 224, which can also be referred to simply as the RSA detector 224, uses signals and/or data obtained from the optical sensor 122 to detect respiratory sinus arrhythmia (RSA). In accordance with an embodiment, the RR detector 222 and/or the RSA detector 224 can communicate with the HRV detector 220 to estimate RR and/or RSA based on HRV and changes therein, as is known in the art.

The BP detector module 226, which can also be referred to simply as the BP detector 226, uses signals and/or data obtained from the optical sensor 122 and the ECG sensor 128 (which can include the electrode 114) to detect a measure of blood pressure (BP). For example, the BP detector 226 can determine a metric of pulse wave velocity (PWV) from a PPG obtained using the optical sensor 122 and an ECG signal obtained using the ECG sensor and can convert the metric of PWV to a metric of blood pressure. The metric of PWV can be determining by determining a time from a specific feature (e.g., an R-wave) of an obtained ECG signal to a specific feature (e.g., a maximum upward slope, a maximum peak or a dicrotic notch) of a simultaneously obtained PPG signal. The BP detector 226 can then be use one or more well-known equations to convert the metric of PWV to one or more metrics of blood pressure, including, but not limited to, systolic blood pressure (SBP) and diastolic blood pressure (DSP).

The SpO2 detector module 228, which can also be referred to simply as the SpO2 detector 228, uses signals and/or data obtained from the optical sensor 122 to detect blood oxygen saturation (SpO2). In order to enable the SpO2 detector 228 to detect SpO2, the optical sensor alternately emits light of two different wavelengths, typically red (e.g., about 660 nm wavelength) and infrared or near infrared (e.g., about 940 nm wavelength), which light is reflected by user tissue such that a light detector of the optical sensor 122 receives incident light that alternates between red and infrared light. As the light is reflected from tissue, some of the energy is absorbed by arterial and venous blood, tissue and the variable pulsations of arterial blood. An interleaved stream of red and infrared light is received by the light detector of the optical sensor 122. The amplitudes of the red light pulses in the light stream are differently effected by the absorption than the infrared light pulses, with the absorptions levels depending on the SpO2 level of the blood. The SpO2 detector 228 can then be use one or more well-known equations to convert relative values indicative of the amount of red and infrared light detected to values of SpO2.

The activity detector module 230, which can also be referred to simply as the activity detector 230, can determine a type and amount of activity of a user based on information such as, but not limited to, motion data obtained using the motion sensor 132, heart rate as determined by the HR detector 218, an amount of ambient light as determined using the outwardly facing ambient light sensor 110, skin temperature as determined by the skin temperature sensor 130, and time of day. The activity detector module 230 can use motion data, obtained using the motion sensor 132, to determine the number of steps that a user has taken with a specified amount of time (e.g., 24 hours), as well as to determine the distance that a user has walked and/or run within a specified amount of time. Activity metrics can be displayed on the digital display 108 and/or uploaded to a base station (e.g., 252) for further analysis.

The calorie burn detector module 232, which can also be referred to simply as the calorie burn detector 230, can determine a current calorie burn rate and an amount of calories burned over a specified amount of time based on motion data obtained using the motion sensor 132, HR as determined using the HR detector 218, and/or skin temperature as determined using the skin temperature sensor 130. A calorie burn rate and/or an amount of calories burned can be displayed on the digital display 108 and/or uploaded to a base station (e.g., 252) for further analysis.

The power manager module 234, which can also be referred to simply as the power manager 234, can use signals and/or data obtained from one or more of the above described sensors and/or modules to determine when to operate the user-wearable device 102 in a first operational mode, and when to operate the user-wearable device 102 in a second operational mode that consumes more power than the first operational mode. Additional details of when the power manager module 234 may operate the user-wearable device 102, or a portion thereof (e.g., the optical sensor 122), in the first and second operational modes are provided below.

The wireless interface 206 can wireless communicate with a base station (e.g., 252), which as mentioned above, can be a mobile phone, a tablet computer, a PDA, a laptop computer, a desktop computer, or some other computing device that is capable of performing wireless communication. The wireless interface 206, and more generally the user wearable device 102, can communicate with a base station 252 using various different protocols and technologies, such as, but not limited to, Bluetooth™, Wi-Fi, ZigBee or ultrawideband (UWB) communication. In accordance with an embodiment, the wireless interface 206 comprises telemetry circuitry that include a radio frequency (RF) transceiver electrically connected to an antenna (not shown), e.g., by a coaxial cable or other transmission line. Such an RF transceiver can include, e.g., any well-known circuitry for transmitting and receiving RF signals via an antenna to and from an RF transceiver of a base station 252.

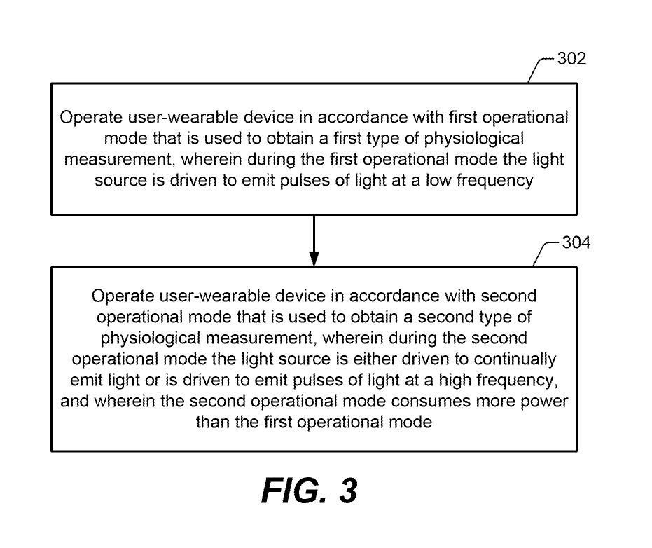

The user-wearable device 102 draws current from its battery 210, and thereby consumes power, when it drives the light source(s) of the optical sensor 122, as well as when it samples the signal(s) (e.g., a PPG signal) produced using the light detector(s) of the optical sensor 122. Where the battery 210 is a rechargeable type of battery, the more power consumed, the more often the battery 210 must be recharged. Where the battery 210 is a non-rechargeable type of battery, the more power consumed, the more often the battery 210 must be replaced with a new battery. In accordance with specific embodiments of the present technology, described below with reference to FIG. 3, power consumption is reduced by selectively changing an operational mode of the optical sensor 122 in dependence on the type of physiological measurement that the user-wearable device 102 wants to obtain. More specifically, in accordance with an embodiment, when the user-wearable device 102 is to obtain a first type of physiological measurement the user-wearable device 102 is operated in accordance with a first operational mode during which the light source(s) of the optical sensor 122 is driven to emit pulses of light at a low frequency, and when the user-wearable device 102 is to obtain a second type of physiologic measurement the user-wearable device is operated in accordance with a second operational mode during which the light source(s) of the optical sensor 102 is either driven to continually emit light or is driven to emit pulses of light at a high frequency. In accordance with an embodiment, during the first operational mode a light detection signal, produced using the light detector of the optical sensor 122, is sampled at a low frequency. By contrast, during the second operational mode the light detection signal, produced using the light detector of the optical sensor 122, is sampled at a high frequency. The terms “low frequency” and “high frequency” as used herein are relative terms that are used to indicate that the “high frequency” is at least one order of magnitude greater (i.e., at least 10× greater) than the “low frequency”. In embodiments where the “high frequency” is more than one order of magnitude greater the “low frequency”, such a distinction will be expressed.

The second operational mode consumes more power than the first operational mode. Accordingly, to conserve power, the second operational mode is preferably only used when the type of physiologic measurement that is to be obtained requires a relatively high temporal resolution, and the first operational mode is used when the type of physiologic measurement that is to be obtained requires only a relatively low temporal resolution. For example, if the physiological measurement to be obtained is HR, then a sufficiently accurate measure of HR can be obtained by operating the optical sensor 122 in the first operational mode that consumes less power than the second operational mode. On the other hand, for example, if the physiological measurement to be obtained is HRV, then the optical sensor 122 is operated in the second operational mode that consumes more power than the first operational mode, because HRV requires a relatively high temporal resolution. For another example, where the physiological measurement is a respiratory sinus arrhythmia (RSA) level, the optical sensor 122 is preferably operated in the second operational mode that consumes more power than the first operational mode, because a measure of RSA level also requires a relatively high temporal resolution.

Another example of a physiologic measurement that can be obtained using the first operational mode is oxygen saturation (SpO2) level. As explained above, in order to obtain measures of SpO2 level, the optical sensor 122 can include one or more light emitting elements (e.g., one or more LEDs) that emit red light and one or more light emitting elements that emit infrared or near infrared light. In accordance with an embodiment, during the first operational mode, the light emitting element(s) that emit red light and the light emitting element(s) that emit infrared or near infrared light are alternately driven such that each of the light emitting elements is driven at the low frequency. In other words, a measure of SpO2 level can be obtained while the optical sensor, or at least the light source of the optical sensor, is operated in the first operational mode.

In accordance with an embodiment, the during the first operational mode, the low frequency at which the light source is driven to emit pulses of light is no greater than 1 kHz, and the high frequency at which the light source is driven during the second operational mode is at least one order of magnitude greater than the low frequency at which the light source is driven to emit pulses of light during the first operational mode. Stated another way, the low frequency at which the light source is driven to emit pulses of light during the first operational mode is at least one order of magnitude less than the high frequency at which the light source is driven during the second operational mode. For example, if during the second operational mode that light source is driven to emit pulses at a frequency of 10 kHz, then the low frequency at which the light source is driven to emit pulses of light during the first operational mode is no greater than 1 kHz. More preferably, during the first operational mode, the low frequency at which the light source is driven to emit pulses of light is no greater than 100 Hz, and during the second operational mode the high frequency at which the light source is driven to emit pulses of light is at least 10 kHz. In this latter example, the high frequency is two orders of magnitude greater than the low frequency.

FIG. 3 is a high level flow diagram that is used to summarize methods according to certain embodiments of the present technology. Referring to FIG. 3, step 302 involves operating the user-wearable device in accordance with a first operational mode that is used to obtain a first type of physiological measurement, wherein during the first operational mode the light source is driven to emit pulses of light at a low frequency. Still referring to FIG. 3, step 304 involves operating the user-wearable device in accordance with a second operational mode that is used to obtain a second type of physiological measurement, wherein during the second operational mode the light source is either driven to continually emit light or is driven to emit pulses of light at a high frequency, and wherein the second operational mode consumes more power than the first operational mode. Step 302 can be performed during a first period of time, and step 304 can be performed during a second period of time that is after (or before) the first period of time. As explained above, the low frequency at which the light source is driven to emit pulses of light during the first operational mode is at least one order of magnitude less than the high frequency at which the light source is driven during the second operational mode.

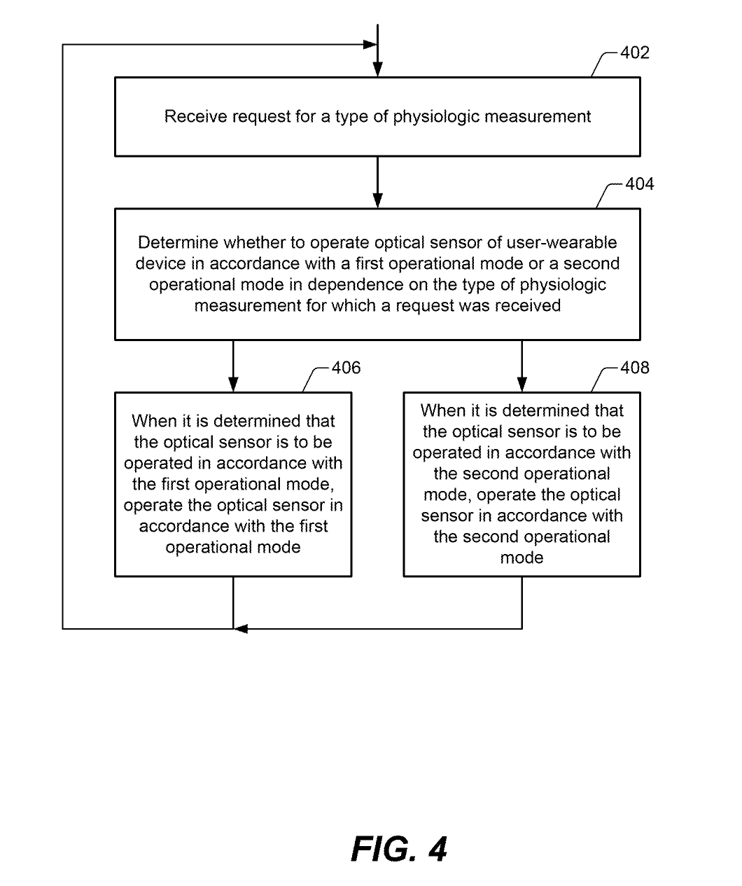

FIG. 4 is another high level flow diagram that is used to summarize methods according to certain embodiments of the present technology. Referring to FIG. 4, step 402 involves receiving a request for a type of physiologic measurement. For example, such a request can be sent from one module to another module within the microcontroller 202, but is not limited thereto. Exemplary types of physiologic measurements include, HR, HRV, RR, RSA, BP and SpO2, but are not limited thereto. Accordingly, step 402 can include receiving a request for one of HR, HRV, RR, RSA, BP and SpO2, but is not limited thereto. At step 404 there is a determination of whether an optical sensor (e.g., 122) of a user-wearable device (e.g., 102) is to be operated in accordance with a first operational mode or a second operational mode in dependence on the type of physiologic measurement for which a request was received at step 402. As indicated at step 406, when it is determined that the optical sensor is to be operated in accordance with the first operational mode, the optical sensor is operated accordingly. As indicated at step 408, when it is determined that the optical sensor is to be operated in accordance with the second operational mode, the optical sensor is operated accordingly. As was described above in additional detail, the first operational mode consumes less power than the second operational mode. Accordingly, the first operational mode can be used to save power compared to if the second operational mode were always used regardless of the type of physiologic measurement that is requested or otherwise needed.

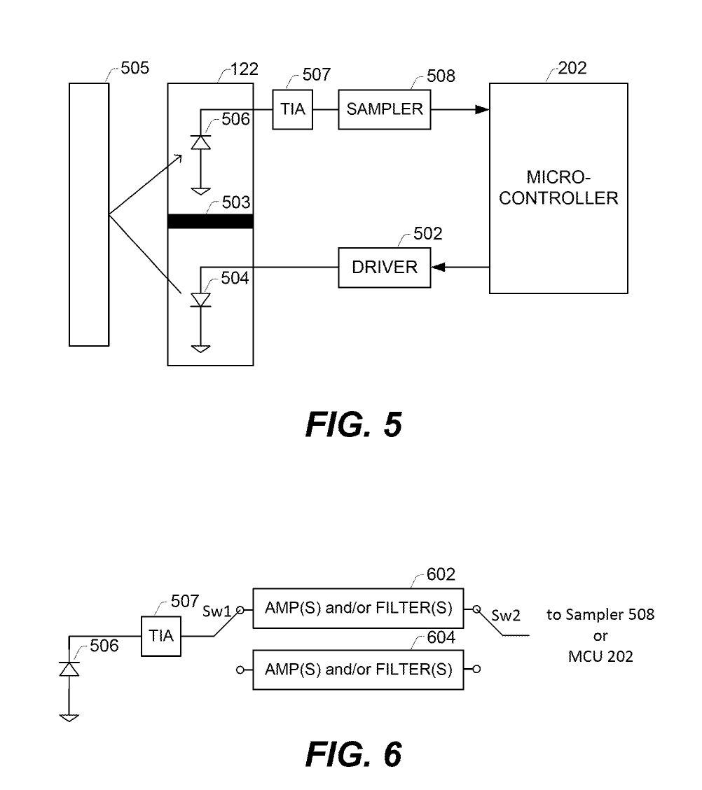

In order to further conserve power, in addition to using a lower light emission frequency during the first operational mode than during the second operational mode, a sampling rate used during the first operational mode can be lower than a sampling rate during the second operational mode. This can be better understood with reference to the block diagram of FIG. 5, which includes some additional details of the optical sensor 122, according to an embodiment.

Referring to FIG. 5, the optical sensor is shown as including a light source 504 and a light detector 506. The light source 504, as mentioned above, can include one or more LED, incandescent lamp or laser diode, but is not limited thereto. The light detector 506 can include one or more one or more photoresistor, photodiode, phototransistor, photodarlington or avalanche photodiode, but is not limited thereto. A driver 502, whose timing is controlled by the microcontroller 202, drives the light source 504 to emit light at a low frequency, a high frequency, or continually. The light detector 506 generates a signal (e.g., a current) that is indicative of the intensity and/or phase of the light incident on the light detector 506. Where the signal generated by the light detector 506 is a current signal, a transimpedance amplifier (TIA) 507 can be used to convert the current signal to a voltage signal. Further analog circuitry, not specifically shown in FIG. 5, can be used to perform analog signal filtering, and/or analog signal amplification of a signal produced by the one or more light detecting elements of the light detector 506. Still referring to FIG. 5, a sampler 508 is shown as sampling the light detection signal produced using the light detector 506. The sampler 508 can alternatively be implemented within and by the microcontroller 202. Element 503 is an opaque barrier that optically isolates the light source 504 from the light detector. As can be appreciated from FIG. 5, the light detector 506 detects light emitted by the light source 504 that reflects off of an object 505 and is incident on the light detector 506.

FIG. 6 is a block diagram that is used to illustrate that different analog signal processing circuitry can be used to amplify and/or filter a light detection signal depending upon an operational mode. More specifically, FIG. 6 illustrates that different analog signal processing circuitry can be used to amplify and/or filter the light detection signal produced using the light detector 506 of the optical sensor 122, depending upon whether the user-wearable device (or the optical sensor thereof) is operating in accordance with its first operational mode or its second operational mode. As explained above, during the first operational mode the light source (e.g., 504 in FIG. 5) is driven to emit pulses of light at a low frequency, and during the second operational mode the light source (e.g., 504 in FIG. 5) is either driven to continually emit light or is driven to emit pulses of light at a high frequency. In FIG. 6, the same TIA 507 is shown as being used regardless of whether the device is operating in the first or second operational mode. Alternatively, the amplification and filter circuitry 602 can includes its own TIA, and the amplification and filter circuitry 604 can includes its own TIA.

When operating in the first operational mode, a switch Sw1 provides the output of the TIA to the amplification and filter circuitry 602. The amplification and filter circuitry 602 can, for example, use an integrating, smoothing or similar type of filter to convert a discontinuous light detection signal to a continuous light detection signal prior to the sampling of such signal. The amplification and filter circuitry 602 can, for example, also include a bandpass filter to filter out frequencies that are not of interest prior to the sampling of the signal. For example, the bandpass frequency range can be from 0.5 Hz to 4 Hz, but is not limited thereto. Additionally, the amplification and filter circuitry 602 can include one or more variable gain amplifier(s) (VGAs) and/or fixed gain amplifier(s) to amplify the light detection signal prior to its sampling. Appropriate amplification may depend on a dynamic range of an analog-to-digital (ADC) within the microcontroller 202 or upstream thereof (but not shown). When operating in the second operational mode, the switch Sw1 provides the output of the TIA to the amplification and filter circuitry 604. Depending upon the operation mode, a switch Sw2 provides the light detection signal (either from the amplification and filter circuitry 602, or from the amplification and filter circuitry 604) to the sampler 508, or alternatively, directly to the microcontroller (e.g., where the function of the sampler is implemented within and by the microcontroller 202).

If the light source (e.g., 504 in FIG. 5) is driven to continually emit light during the second operational mode, then the light detection signal should be a continuous signal, and there is no need for the amplification and filter circuitry 604 to convert a discontinuous light detection signal to a continuous light detection. If the light source (e.g., 504 in FIG. 5) is driven to emit light at a high frequency during the second operational mode, then the amplification and filter circuitry 604 may convert a discontinuous light detection signal to a continuous light detection using a similar filter that is used by the amplification and filter circuitry 602, but which has a different RC time constant than the similar filter used by the amplification and filter circuitry 602. The amplification and filter circuitry 604 can, for example, also include a bandpass filter to filter out frequencies that are not of interest prior to the sampling of the signal. For example, the bandpass frequency range can be from 0.5 Hz to 4 Hz, but is not limited thereto. Additionally, the amplification and filter circuitry 602 can include one or more variable gain amplifier(s) and/or fixed gain amplifier(s) to amplify the light detection signal prior to its sampling. Where the two analog signal paths include common circuitry, the analog signal processing circuitry can be designed such that at least some of the same circuitry is used regardless of whether the device is operating in the first or second operational modes. More generally, at least some analog filtering and/or amplification circuitry can be specifically used when the device is operating in the first operational mode, and at least some different analog filtering and/or amplification circuitry can be specifically used when the device is operating in the second operational mode.

In accordance with an embodiment, during the first operational mode, the low frequency at which the light detection signal is sampled is at least one order of magnitude less than the high frequency at which the light detection signal is sampled during the second operational mode. For example, if during the second operational mode that light detection signal is sampled at a frequency of 10 kHz, then the low frequency at which the light detection signal is sampled during the first operational mode is no greater than 1 kHz. More preferably, during the first operational mode, the low frequency at which the light detection signal is sampled is no greater than 100 Hz, and during the second operational mode the high frequency at which the light detection signal is sampled is at least 10 kHz.

In an embodiment, the low frequency at which the light detection signal is sampled during the first operational mode can be the same as the low frequency at which the light source is driven to emit pulses of light during the first operational mode. Alternatively, the low frequency at which the light detection signal is sampled during the first operational mode can differ from the low frequency at which the light source is driven to emit pulses of light during the first operational mode. In an embodiment, the high frequency at which the light detection signal is sampled during the second operational mode can be the same as the high frequency at which the light source is driven to emit pulses of light during the second operational mode. Alternatively, the high frequency at which the light detection signal is sampled during the second operational mode can differ from the high frequency at which the light source is driven to emit pulses of light during the second operational mode. The frequencies used to drive the light source can be referred to as drive frequencies, and the frequencies used to sample the light detection signals produced using the light detector can be referred to as the sample frequencies. Accordingly, during the first operational mode, the low sample frequency can be the same as the low drive frequency, or can be different than the low drive frequency. During the second operational mode, the high sample frequency can be the same as the high drive frequency, or can be different than the high drive frequency.

Referring briefly back to FIGS. 1A and 1B, the user-wearable device 102 was generally shown and described as being a wrist-wearable device that can be strapped to a user's wrist, or another portion of a user's arm. However, embodiments described herein should not be limited to use with wrist-wearable devices. For example, embodiments described herein can also be used with chest-wearable, head-wearable or leg-wearable devices, but are not limited thereto. In other words, the user-wearable devices described herein are not intended to be limited to the form factors shown in the FIGS. and described above. More generally, embodiments of the present technology described herein can be used with most any user-wearable device that includes a light source and a light detector and that is configured to obtain at least two different types of physiological measurements using the light source and the light detector, wherein one of the types of physiological measurements can be obtained by driving the light source at a lower frequency than the other one of the types of physiological measurements Described herein are methods for use with a battery powered user-wearable device that includes a light source and a light detector and that is configured to obtain at least two different types of physiological measurements using the light source and the light detector. In certain embodiments, the light source and the light detector can be collectively referred to as an optical sensor. In accordance with an embodiment, during a first period of time, the user-wearable device is operated in accordance with a first operational mode that is used to obtain a first type of physiological measurement, wherein during the first operational mode the light source is driven to emit pulses of light at a low frequency. During a second period of time, the user-wearable device is operated in accordance with a second operational mode that is used to obtain a second type of physiological measurement. During the second operational mode the light source is either driven to continually emit light or is driven to emit pulses of light at a high frequency. Accordingly, the second operational mode consumes more power than the first operational mode. The first type of physiological measurement can be, e.g., HR, and the second type of physiological measurement can be, e.g., HRV. Additionally, in accordance with an embodiment, during the first operational mode a light detection signal, produced using the light detector, can be sampled at a low frequency. During the second operational mode the light detection signal, produced using the light detector, can be sampled at a high frequency.

In accordance with an embodiment, during the first operational mode, the low frequency at which the light source is driven to emit pulses of light is no greater than 1 kHz. During the second operational mode, the light source is driven to emit pulses of light at a high frequency that is at least one order of magnitude greater than the low frequency at which the light source is driven to emit pulses of light during the first operational mode. In a specific embodiment, during the first operational mode, the low frequency at which the light source is driven to emit pulses of light is no greater than 100 Hz, and during the second operational mode, the high frequency at which the light source is driven to emit pulses of light is at least 10 kHz.

In accordance with an embodiment, the light source includes a first light emitting element that emits light of a first wavelength when driven and a second light emitting element that emits light of a second wavelength when driven. During the first operational mode, the first and second light emitting elements of the light source can be alternately driven such that each of the first and second light emitting elements is driven at the low frequency. In such an embodiment, the first type of physiological measurement can be, e.g., HR or SpO2 level, and the second type of physiological measurement can be, e.g., HRV or RSA level.

More generally, in accordance with certain embodiments, the first type of physiological measurement can be selected from the group consisting of HR and SpO2 level, and the second type of physiological measurement can be selected from the group consisting of HRV, RSA level, a measurement of BP, and RR.

Certain embodiments involve receiving a request for a type of physiologic measurement, and determining whether to operate the optical sensor of a user-wearable device in accordance with a first operational mode or a second operational mode in dependence on the type of physiologic measurement for which the request was received. When it is determined that the optical sensor is to be operated in accordance with the first operational mode, the optical sensor is operated in accordance with the first operational mode. When it is determined that the optical sensor is to be operated in accordance with the second operational mode, the optical sensor can be operated in accordance with the second operational mode, which consumes more power than the first operational mode. In certain embodiments, operating the optical sensor in accordance with the first operational mode includes driving a light source of the optical sensor to emit pulses of light at a frequency of no greater than 100 Hz, and sampling a light detection signal, produce using a light detector of the optical sensor, at a frequency of no greater than 100 Hz. In certain embodiments, operating the optical sensor in accordance with the second operational mode includes driving a light source of the optical sensor to emit pulses of light at a frequency of at least 10 kHz, and sampling a light detection signal, produce using a light detector of the optical sensor, at a frequency of at least 10 kHz.

Certain embodiments of the present technology are directed to a user-wearable device including a battery, a light source that emits light in response to being driven, and a light detector that detects light emitted by the light source that reflects off of an object and is incident on the light detector. The user-wearable device can also include a power manager that controls when the light source is driven in accordance with a first operational mode during which pulses of light can be emitted at a low frequency, and when the light source is driven in accordance with a second operational mode during which light is continually emitted or pulses of light are emitted at a high frequency, wherein the second operational mode consumes more power from the battery than the first operational mode. The user-wearable device can also include a first module that obtains a first type of physiological measurement (e.g., HR) when the light source is driven in accordance with the first operational mode, and a second module that obtains a second type of physiological measurement (e.g., HRV) when the light source is driven in accordance with the second operational mode. The battery provides power to drive the light during the first and second operational modes. Less power is consumed from the battery during the first operational mode compared to during the second operational mode. Additionally, during the first operational mode a light detection signal, produced using the light detector, can be sampled at a low frequency. During the second operational mode, a light detection signal, produced using the light detector, is sampled at a high frequency. For example, during the first operational mode, the low frequency at which the light source is driven to emit pulses of light is not greater than 1 kHz. During the second operational mode, the light source is driven to emit pulses of light at a high frequency that is at least one order of magnitude greater than the low frequency at which the light source is driven to emit pulses of light during the first operational mode. For an even more specific example, during the first operational mode, the low frequency at which the light source is driven to emit pulses of light is no greater than 100 Hz, and during the second operational mode, the high frequency at which the light source is driven to emit pulses of light is at least 10 kHz.

In accordance with certain embodiments, the light source of the user-wearable device includes a first light emitting element that emits light of a first wavelength when driven and a second light emitting element that emits light of a second wavelength when driven. During the first operational mode, the first and second light emitting elements of the light source are alternately driven such that each of the first and second light emitting elements is driven at the low frequency. The first type of physiological measurement can be, e.g., HR or SpO2 level. The second type of physiological measurement can be, e.g., HRV or RSA level.

In accordance with an embodiment, the user-wearable can including a housing having a front side and a back side, with the battery within the housing. The user-wearable device can also include a band that straps the housing to a person's wrist. The user-wearable device can also a digital display on the front side of the housing, and an optical sensor on or adjacent the back side of the housing. The optical sensor can include a light source that emits light in response to being driven, and a light detector that detects light emitted by the light source that reflects off of an object and is incident on the light detector. When the optical sensor is in a first operational mode, the light source is driven to emit pulses of light are at a first drive frequency. When the optical sensor is in a second operational mode, the light source can be driven to emit pulses of light at a second drive frequency that is at least one order of magnitude greater than the first drive frequency. A first type of physiological measurement can be obtained when the optical sensor is operated in accordance with the first operational mode. A second type of physiological measurement can be obtained when the optical sensor is operated in accordance with the second operational mode. The battery provides power to drive the light source of the optical sensor during the first and second operational modes. Less power is consumed from the battery during the first operational mode compared to during the second operational mode. In certain embodiments, when the optical sensor is in the first operational mode, a light detection signal produced using the light detector of the optical sensor is sampled at a first sampling frequency. When the optical sensor is in the second operational mode, the light detection signal produced using the light detector of the optical sensor is sampled at a second sampling frequency that is at least one order of magnitude greater than the first sampling frequency.

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen to best explain the principles of the technology and its practical application to thereby enable others skilled in the art to best utilize the technology in various embodiments and with various modifications as are suited to the particular use contemplated. It is intended that the scope of the technology be defined by the claims appended hereto. While various embodiments have been described above, it should be understood that they have been presented by way of example, and not limitation. It will be apparent to persons skilled in the relevant art that various changes in form and detail can be made therein without departing from the spirit and scope of the technology. The breadth and scope of the present technology should not be limited by any of the above-described exemplary embodiments, but should be defined only in accordance with the following claims and their equivalents.

What is claimed is:

1. A method for use with a battery powered user-wearable device that includes a light source and a light detector and that is configured to obtain at least two different types of physiological measurements using the light source and the light detector, the method comprising:
(a) during a first period of time, operating the user-wearable device in accordance with a first operational mode that is used to obtain a first type of physiological measurement, wherein during the first operational mode the light source is driven to emit pulses of light at a low frequency; and
(b) during a second period of time, operating the user-wearable device in accordance with a second operational mode that is used to obtain a second type of physiological measurement, wherein during the second operational mode the light source is either driven to continually emit light or is driven to emit pulses of light at a high frequency, and wherein the second operational mode consumes more power than the first operational mode.

2. The method of claim 1, wherein:
the first type of physiological measurement comprises heart rate (HR); and
the second type of physiological measurement comprises heart rate variability (HRV).

3. The method of claim 1, wherein:
during the first operational mode a light detection signal, produced using the light detector, is sampled at a low frequency;
during the second operational mode the light detection signal, produced using the light detector, is sampled at a high frequency.

4. The method of claim 1, wherein:
during the first operational mode a light detection signal, produced using the light detector, is filtered and/or amplified using first analog circuitry; and
during the second operational mode a light detection signal, produced using the light detector, is filtered and/or amplified using second analog circuitry at least a portion of which differs from the first analog circuitry.

5. The method of claim 1, wherein:
during the first operational mode, the low frequency at which the light source is driven to emit pulses of light is no greater than 1 kHz.

6. The method of claim 5, wherein:
during the second operational mode, the light source is driven to emit pulses of light at a high frequency that is at least one order of magnitude greater than the low frequency at which the light source is driven to emit pulses of light during the first operational mode.

7. The method of claim 6, wherein:
during the first operational mode, the low frequency at which the light source is driven to emit pulses of light is no greater than 100 Hz; and
during the second operational mode, the high frequency at which the light source is driven to emit pulses of light is at least 10 kHz.

8. The method of claim 1, wherein the light source includes a first light emitting element that emits light of a first wavelength when driven and a second light emitting element that emits light of a second wavelength when driven, and wherein:
during the first operational mode, the first and second light emitting elements of the light source are alternately driven such that each of the first and second light emitting elements is driven at the low frequency; and
the first type of physiological measurement is selected from the group consisting of heart rate (HR) and oxygen saturation (SpO₂) level; and
the second type of physiological measurement is selected from the group consisting of heart rate variability (HRV) and respiratory sinus arrhythmia (RSA) level.

9. The method of claim 1, wherein:
the first type of physiological measurement is selected from the group consisting of heart rate (HR) and oxygen saturation (SpO₂) level; and
the second type of physiological measurement is selected from the group consisting of heart rate variability (HRV), respiratory sinus arrhythmia (RSA) level, a measurement of blood pressure (BP), and respiratory rate (RR).

10. A method for use with a battery powered user-wearable device that includes an optical sensor, the method comprising:
(a) receiving a request for a type of physiologic measurement;
(b) determining whether to operate the optical sensor of the user-wearable device in accordance with a first operational mode or a second operational mode in dependence on the type of physiologic measurement for which the request was received;
(c) when it is determined that the optical sensor is to be operated in accordance with the first operational mode, operating the optical sensor in accordance with the first operational mode; and
(d) when it is determined that the optical sensor is to be operated in accordance with the second operational mode, operating the optical sensor in accordance with the second operational mode, which consumes more power than the first operational mode.

11. The method of claim 10, wherein:
at step (c) operating the optical sensor in accordance with the first operational mode includes
(c.1) driving a light source of the optical sensor to emit pulses of light at a frequency of no greater than 100 Hz, and
(c.2) sampling a light detection signal, produce using a light detector of the optical sensor, at a frequency of no greater than 100 Hz; and
at step (d) operating the optical sensor in accordance with the second operational mode includes
(c.1) driving a light source of the optical sensor to emit pulses of light at a frequency of at least 10 kHz, and
(c.2) sampling a light detection signal, produce using a light detector of the optical sensor, at a frequency of at least 10 kHz.

13. The method of claim 12, wherein:
at step (c) operating the optical sensor in accordance with the first operational mode includes filtering and/or amplifying the light detection signal, prior to the sampling, using first analog circuitry; and
at step (d) operating the optical sensor in accordance with the second operational mode includes filtering and/or amplifying the light detection signal, prior to the sampling, using second analog circuitry at least a portion of which differs from the first analog circuitry.

14. A user-wearable device, comprising:
a battery;
a light source that emits light in response to being driven;
a light detector that detects light emitted by the light source that reflects off of an object and is incident on the light detector;
a power manager that controls
when the light source is driven in accordance with a first operational mode during which pulses of light are emitted at a low frequency, and
when the light source is driven in accordance with a second operational mode during which light is continually emitted or pulses of light are emitted at a high frequency, wherein the second operational mode consumes more power from the battery than the first operational mode;
a first module that obtains a first type of physiological measurement based on a light detection signal produced using the light detector when the light source is driven in accordance with the first operational mode; and
a second module that obtains a second type of physiological measurement based on a light detection signal produced using the light detector when the light source is driven in accordance with the second operational mode;
wherein the battery provides power to drive the light during the first and second operational modes; and
wherein less power is consumed from the battery during the first operational mode compared to during the second operational mode.

15. The user-wearable device of claim 14, wherein:
the first type of physiological measurement comprises heart rate (HR); and
the second type of physiological measurement comprises heart rate variability (HRV).

16. The user-wearable device of claim 14, wherein:
during the first operational mode a light detection signal, produced using the light detector, is sampled at a low frequency;
during the second operational mode a light detection signal, produced using the light detector, is sampled at a high frequency.

17. The user-wearable device of claim 14, wherein:
during the first operational mode, the low frequency at which the light source is driven to emit pulses of light is not greater than 1 kHz.

18. The user-wearable device of claim 16, wherein:
during the second operational mode, the light source is driven to emit pulses of light at a high frequency that is at least one order of magnitude greater than the low frequency at which the light source is driven to emit pulses of light during the first operational mode.

19. The user-wearable device of claim 18, wherein:
during the first operational mode, the low frequency at which the light source is driven to emit pulses of light is no greater than 100 Hz; and
during the second operational mode, the high frequency at which the light source is driven to emit pulses of light is at least 10 kHz.

20. The user-wearable device of claim 18, wherein:
the light source includes a first light emitting element that emits light of a first wavelength when driven and a second light emitting element that emits light of a second wavelength when driven:
during the first operational mode, the first and second light emitting elements of the light source are alternately driven such that each of the first and second light emitting elements is driven at the low frequency; and
the first type of physiological measurement is selected from the group consisting of heart rate (HR) and oxygen saturation (SpO₂) level; and
the second type of physiological measurement is selected from the group consisting of heart rate variability (HRV) and respiratory sinus arrhythmia (RSA) level.

21. The user-wearable device of claim 14, further comprising:
first analog circuitry that filters and/or amplifies the light detection signal produced by the light detector during the first operational mode; and
second analog circuitry that filters and/or amplifies the light detection signal produced by the light detector during the second operational mode.

22. A user-wearable device, comprising:
a housing having a front side and a back side;
a battery within the housing;
a band that straps the housing to a person's wrist;
a digital display on the front side of the housing; and
an optical sensor, on or adjacent the back side of the housing, including a light source that emits light in response to being driven, and a light detector that detects light emitted by the light source that reflects off of an object and is incident on the light detector;
wherein when the optical sensor is in a first operational mode, the light source is driven to emit pulses of light are at a first drive frequency; and
wherein when the optical sensor is in a second operational mode, the light source is driven to emit pulses of light at a second drive frequency that is at least one order of magnitude greater than the first drive frequency.

23. The user-wearable device of claim 22, wherein:
a first type of physiological measurement is obtained when the optical sensor is operated in accordance with the first operational mode; and
a second type of physiological measurement is obtained when the optical sensor is operated in accordance with the second operational mode;
the battery provides power to drive the light source of the optical sensor during the first and second operational modes; and
less power is consumed from the battery during the first operational mode compared to during the second operational mode.

24. The user-wearable device of claim 22, wherein:
wherein when the optical sensor is in the first operational mode, a light detection signal produced using the light detector of the optical sensor is sampled at a first sampling frequency; and
wherein when the optical sensor is in the second operational mode, the light detection signal produced using the light detector of the optical sensor is sampled at a second sampling frequency that is at least one order of magnitude greater than the first sampling frequency.
